(19) United States
(12) Patent Application Publication
(10) Pub. No.: US 2022/0015954 A1
(43) Pub. Date: Jan. 20, 2022

(54) ROUND TIP VAGINAL INSERT SYSTEM AND METHOD OF USING THE SAME

(71) Applicant: Darvyaire Turner, Chicago, IL (US)

(72) Inventor: Darvyaire Turner, Chicago, IL (US)

(21) Appl. No.: 17/490,282

(22) Filed: Sep. 30, 2021

Related U.S. Application Data
(63) Continuation-in-part of application No. 15/913,624, filed on Mar. 6, 2018, now abandoned.
(60) Provisional application No. 62/467,325, filed on Mar. 6, 2017.

(51) Int. Cl. A61F 13/26

(52) U.S. Cl. CPC A61F 13/266; A61F 2013/15105

(57) ABSTRACT

A feminine hygiene product may include an outer receptacle including a bulb shaped head including a plurality of flaps and defining a plurality of slits therein, the outer receptacle being constructed and arranged for comfortable insertion within a vaginal cavity. The feminine hygiene product may include a loading cylinder portion and an absorbent element, the loading cylinder being constructed and arranged to slidingly engage with the outer receptacle and push the absorbent element through the outer receptacle and through the plurality of flaps such that the absorbent element may be disposed within at least a portion of a vaginal cavity to absorb fluids therein. The absorbent element may include a cord or other means for subsequent removal of the absorbent element from the vaginal cavity.

### CROSS-REFERENCE TO RELATED APPLICATIONS

This application is a continuation-in-part and claims the benefit of U.S. Provisional Application Ser. No. 62/467,325, filed Mar. 6, 2017, and U.S. patent application Ser. No. 15/913,624, filed Mar. 6, 2018, which are incorporated herein by reference.

### TECHNICAL FIELD

The embodiments generally relate to feminine hygiene products.

### BACKGROUND

Feminine hygiene products inserted into the vaginal cavity may serve the purpose of collecting fluids discharged during menstruation. A variety of solutions exist for the collection of discharged fluids and tissue debris from the uterus that occurs during menstruation. However, there is no effective solution for collecting bodily fluids remaining in the vaginal cavity post sexual intercourse. Liners may be used to capture post-coital fluids but this solution fails to capture post-coital fluids leaking from the vaginal cavity resulting in unpleasant feeling, odors, unwanted fluids, and messy surroundings after sexual intercourse. Undesirable fluids may include, but are not limited to, natural or artificial fluids.

A variety of feminine hygiene products in the market such as tampons suffer from risks of causation of toxic shock syndrome (TSS). Higher-absorbency tampons may increase the risk of TSS. Additionally, tampons are not suitable for use outside of a woman's menstruation cycle diminishing the product's usefulness in post coital situations. However, the embodiments of the present disclosure provide several advantages such as reduction of the risk of TSS. Additionally, embodiments of the present disclosure provide several advantages including providing contraceptive protection, a barrier, a spermicide, effective absorption, and STI prevention. A product for the hygienic and convenient collection of fluids post sexual intercourse may overcome the deficiencies in the current art.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments described herein provide a feminine hygiene product for the convenient and hygienic removal of fluids from the vagina, cervix, cervical external orifice, cervical canal, cervical internal canal, or uterine cavity.

The embodiments described herein additionally provide a method of using a feminine hygiene product for the convenient and hygienic removal of fluids from the vagina, cervix, cervical external orifice, cervical canal, cervical internal canal, or uterine cavity.

The embodiments described herein provide a feminine hygiene product that may include an outer receptacle including a bulb shaped head including a plurality of flaps and defining a plurality of slits therein, the outer receptacle being constructed and arranged for comfortable insertion within a vaginal cavity. The feminine hygiene product may include a loading cylinder portion and an absorbent element, the loading cylinder being constructed and arranged to slidingly engage with the outer receptacle and push the absorbent element through the outer receptacle and through the plurality of flaps such that the absorbent element may be disposed within at least a portion of a vaginal cavity to absorb fluids therein. The absorbent element may include a cord or other means for subsequent removal of the absorbent element from the vaginal cavity.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1A illustrates an example schematic of an apparatus comprising an embodiment of an outer receptacle consistent with this disclosure;

FIG. 1B illustrates an example schematic of an apparatus comprising a loading cylinder consistent with this disclosure;

FIG. 2 illustrates an example schematic of a system comprising an outer receptacle, a loading element, and an absorbent element consistent with this disclosure;

FIG. 3 illustrates an example schematic of an apparatus comprising an embodiment of an outer receptacle consistent with this disclosure; and

FIG. 4 illustrates an example schematic of a method of using the system consistent with this disclosure.

### DETAILED DESCRIPTION

The specific details of the single embodiment or variety of embodiments described herein are to the described apparatus. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the apparatus. Accordingly, the apparatus components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The specific details of the single embodiment or variety of embodiments described herein are set forth in this application. Any specific details of the embodiments are used for demonstration purposes only, and no unnecessary limitation or inferences are to be understood therefrom. Furthermore, as used herein, relational terms, such as “first” and “second,” “top” and “bottom,” and the like, may be used solely to distinguish one entity or element from another entity or element without necessarily requiring or implying any physical or logical relationship, or order between such entities or elements.

In general, the embodiments provided herein relate to a feminine hygiene product that may include an outer receptacle including a bulb shaped head including a plurality of flaps and defining a plurality of slits therein, the outer receptacle being constructed and arranged for comfortable insertion within a vaginal cavity. The outer receptacle may be generally cylindrical and define a receiving passage constructed and arranged to slidably engage with the absorbent element and loading cylinder, wherein the loading cylinder may plunge within the receiving passage to push the absorbent element towards the bulb shaped head of the outer receptacle. The outer receptacle may include a bulb shaped head including a plurality of flaps and defining a plurality of slits therein, the plurality of slits being in operable communication with receiving passage such that when the absorbent element is passed through the receiving passage the absorbent element may apply a force to the plurality of flaps allowing the flaps to flexibly extend away from one another at the plurality of slits such that the bulb shaped head may at least partially open and allow the absorbent element to pass therethrough.

The bulb shaped head may be any of a number of various shapes including, but not limited to, spherical, semi-spherical, tear drop shaped, egg shaped, approximately cone shaped, approximately cube shaped, approximately tetrahedron or square pyramid shaped, approximately cuboid, or any other suitable shape. In general, the bulb shaped head may have a diameter or longest cross-sectional dimension larger than that of the diameter of the generally cylindrical outer receptacle. In general, bulb shaped head may have a cross-sectional area larger than that of the cross-sectional area of the outer receptacle. According to some embodiments, the bulb shaped head.

The feminine hygiene product may include a loading cylinder portion and an absorbent element, the loading cylinder being constructed and arranged to slidingly engage with the receiving passage of the outer receptacle and push the absorbent element through the outer receptacle and through the plurality of flaps such that the absorbent element may be disposed within at least a portion of a vaginal cavity to absorb fluids therein. The loading cylinder may define a loading recess for receiving at least a portion of the absorbent element prior to inserting the absorbent element and loading cylinder into the receiving passage.

The absorbent element may include a cord or other means for subsequent removal of the absorbent element from a vaginal cavity. The absorbent element may be a generally cylindrical absorbent material such as, but not limited to, a sponge, cloth, or similar substance or material. The absorbent element may further include an absorbent sponge, natural cotton, organic cotton, synthetic cotton, cotton, bead, gel, chemical compound, composite element, an absorbent chemical composition, paste, or the like. The absorbent element may be implemented in connection with any other types of contraceptives, medical devices, and feminine hygiene products, such as an intrauterine device (IUD), a birth control pill, a condom, a sponge, a gel, a foam, a cream, a birth control shot, a spermicide, a diaphragm or the like. According to some variations, the absorbent element may include a contraceptive coating or substance therein such that when the absorbent element is disposed within a vaginal cavity, the contraceptive coating or substance may provide additional contraception. The absorbent element may include a contraceptive or a medical chemical for disease prevention. The absorbent element may contain a spermicide such as nonoxynol-9, spermicidal jelly, cream, foam, tablets, suppositories, sponge, film, plant-based spermicides, natural spermicides, organic spermicides, oils, fats, ghee, or the like.

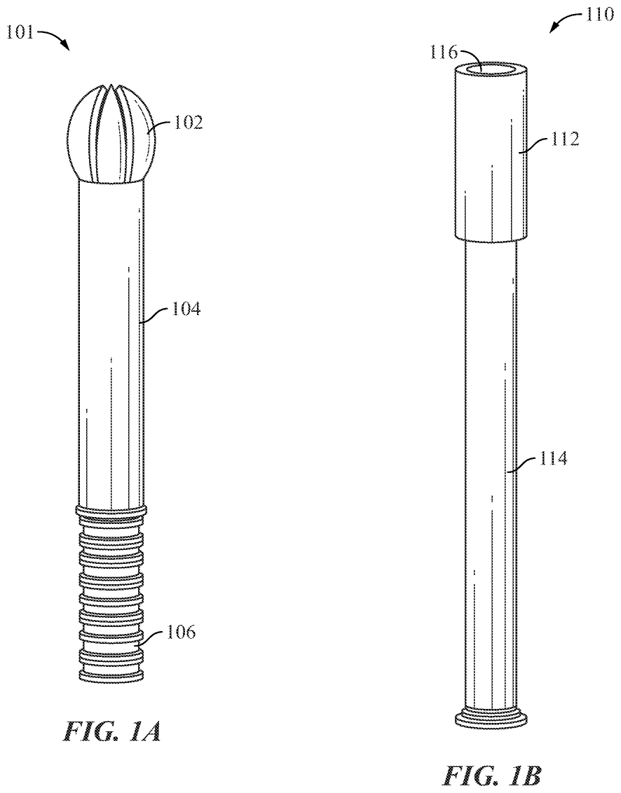

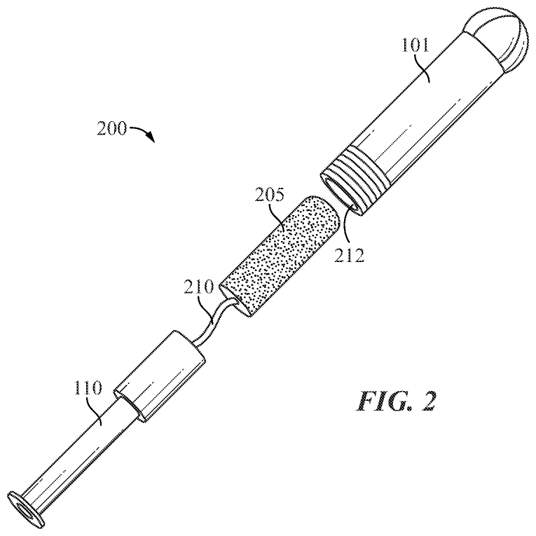

FIGS. 1A and 1B depict one variation of a feminine hygiene product including an outer receptacle 101 including a bulb shaped head 102 having a cross-sectional area larger than that of the cross-sectional area of a cylindrical shaft portion 104, and a threaded base component 106. The outer receptacle 101 may define a receiving passage there in as best seen in FIG. 2. The feminine hygiene product may include a loading cylinder 110 that may include a head 112 defining a loading recess 116 and a base 114. The loading cylinder 110 may be constructed and arranged to slightly engage with the receiving passage of the outer receptacle 101.

Referring to FIG. 2, the feminine hygiene product 200 may include an outer receptacle 101 defining a receiving passage 212 constructed and arranged to receive at least a portion of the loading cylinder 110, and the absorbent element 205. The absorbent element 205 may include a cord or string 210 attached thereto. The absorbent element 205 may be constructed and arranged to at least partially seat within the loading recess of the loading cylinder 110, the loading rest recess being seen best in FIG. 1B.

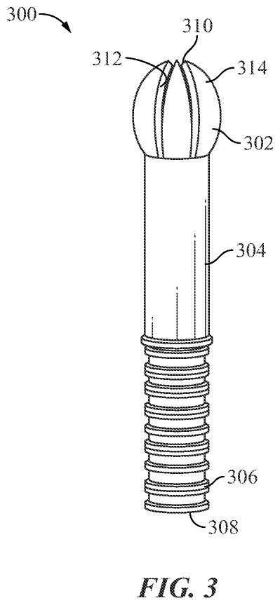

Referring to FIG. 3, the outer receptacle 300 may include a bulb shaped head 302 attached to a cylindrical shaft 304 attached to a threaded base component 306 having a bottom end 308. The bulb shaped had 302 may include an expulsion end 310 including a plurality of flaps 314 and defining a plurality of slits 312 defined between each of the individual flaps in the plurality of flaps 314. The expulsion end 310 may be constructed and arranged to flexibly open via the plurality of flaps 314 when the loading cylinder and absorbent element are pushed through the receiving passage of the outer receptacle 300 such that the absorbent element may be pushed out of the expulsion end 310 of the feminine hygiene product.

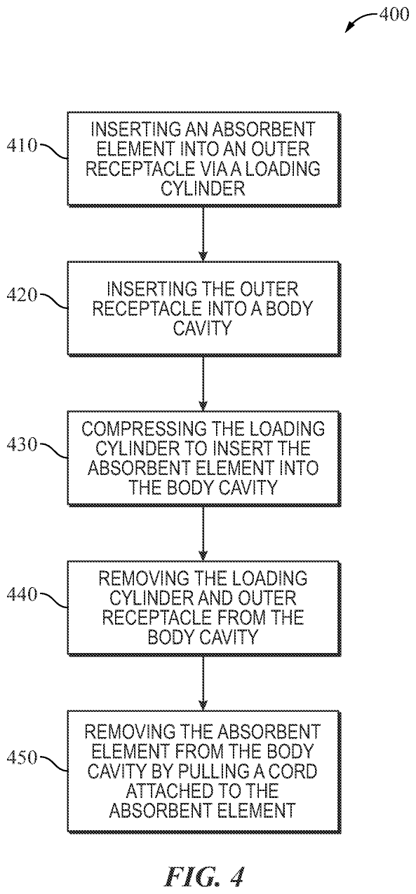

As shown in FIG. 4, in practice and in use 400, the feminine hygiene product may be pre-assembled or may be assembled by inserting the absorbent element and cord within the loading recess of the loading cylinder. The absorbent element, cord, and loading cylinder may be partially seated within the receiving passage of the outers receptacle to complete the assembled feminine hygiene product 410. In use, a portion of the outer receptacle may be inserted into a vaginal cavity 420 and the loading cylinder may be plunged within the receiving passage thereby pushing the absorbent element through the receiving passage and at least partially out of the expulsion end of the outer receptacle 430. The absorbent element may be constructed and arranged to absorb fluids within the vaginal cavity. The outer receptacle and loading cylinder may be removed from the vaginal cavity 440. The cord, affixed to the absorbent element, may be used to remove the absorbent element from the vaginal cavity thereby removing undesirable fluids from the vaginal cavity 450.

The following description of variants is only illustrative of components, elements, acts, products, and methods considered to be within the scope of the invention and are not in any way intended to limit such scope by what is specifically disclosed or not expressly set forth. The components, elements, acts, products, and methods as described herein may be combined and rearranged other than as expressly described herein and are still considered to be within the scope of the invention.

According to variation 1, a device may be configured to be inserted into a vaginal cavity, the device may include an outer receptacle having a bottom end, an expulsion end, and a cylindrical shaft portion disposed between the bottom end and the expulsion end, the cylindrical shaft portion defining a receiving passage portion therethrough extending between the bottom end and the expulsion end wherein the expulsion end includes a bulb shaped head defining a plurality of slits defining a plurality of arc-shaped flexible flap members defining an opening and wherein the outer receptacle and the bulb shaped head member include an ergonomic shape adapted to be slidably inserted into the vaginal cavity with ease and comfort; an absorbent element selected at least from the group consisting of a sponge, bead, gel, chemical compound, chemical composition, or medicament, the absorbent element adapted to conform to the receiving passage portion of the cylindrical shaft portion, the absorbent element being slidably inserted via the bottom open end and the absorbent element includes a pulling cord member; and a loading cylinder member having an ergonomic shape that is slidably engaged with the receiving passage portion for forcing the absorbent element through the receiving passage portion of the cylindrical shaft portion of the outer receptacle and at least partially out of the bulb shaped head member.

Variation 2 may include the device of variation 1 wherein the bulb shaped head has a longest cross-sectional dimension greater than the diameter of the cylindrical shaft portion.

Variation 3 may include the device as in any of variations 1 through 2 wherein the bulb shaped head has a cross-sectional area larger than that of the cross-sectional area of the cylindrical shaft portion

Variation 4 may include the device as in any of variations 1 through 3 wherein the absorbent element is configured to reduce a rate of risk for toxic shock syndrome.

Variation 5 may include the device as in any of variations 1 through 4 wherein the absorbent element includes at least one of a spermicide or contraceptive.

Variation 6 may include the device as in any of variations 1 through 5 wherein the absorbent element includes at least one of a medical chemical or medicament for disease prevention.

Variation 7 may include the device as in any of variations 1 through 6 wherein the device is configured to leave the outer receptacle and the absorbent element intact within the vaginal cavity.

Variation 8 may include the device as in any of variations 1 through 7 wherein the outer receptacle is formed according to at least one of a selective predetermined length of the absorbent element and a selective predetermined radius of the absorbent element wherein the selective predetermined length is directly proportional with the absorbency rate of the absorbent element.

Variation 9 may include the device as in any of variations 1 through 8 wherein the absorbent element is constructed and arranged to absorb at least one fluid at an effective absorption rate.

Variation 10 may include the device as in any of variations 1 through 9 wherein the absorbent element absorbs the fluids at an absorption rate greater than fifty percent.

According to variation 11, a method may include inserting an absorbent element including a cord into a receiving passage defined by an outer receptacle and sliding a loading cylinder within the outer receptacle to position the absorbent element within the receiving passage to provide a feminine hygiene product adapted for insertion within a vaginal cavity; inserting the feminine hygiene product into a cavity; compressing the loading cylinder within the cylindrical shaft portion to expel the absorbent element through the opening defined by the plurality of arc-shaped flexible flap members and into the cavity with a reduced force impact for ease and comfort therein, wherein the absorbent element includes at least one of a sponge, bead, gel, chemical compound, chemical composition, or medicament; and removing the loading cylinder and the outer receptacle and leaving the absorbent element intact within the cavity.

Variation 12 may include the method as in variation 11 further including removing the absorbent element from the cavity by pulling the cord.

Variation 13 may include the method as in any of variations 11 through 12 wherein the absorbent element is constructed and arranged to absorb bodily fluids.

Variation 14 may include the method as in any of variations 11 through 13 wherein the outer receptacle further includes at least one of a bulb shaped head or a threaded base component defining a hand gripping portion.

Variation 15 may include the method as in any of variations 11 through 14 wherein the absorbent element is specifically configured to absorb seminal fluids.

Variation 16 may include the method as in any of variations 11 through 15 wherein the absorbent element further includes at least one of a spermicide, a contraceptive, or a medical chemical for disease prevention.

Variation 17 may include the method as in any of variations 11 through 16 wherein the outer receptacle, the bulb-shaped head member, and the loading cylinder include ergonomic configurations.

According to variation 18, an apparatus adapted for use with a vaginal cavity may include an outer receptacle having an ergonomic configuration, the outer receptacle including a bottom end defining a receiving passage, the bottom end being opposite a bulb shaped head including a plurality of ergonomic arc-shaped members defining an opening therethrough in communication with the receiving passage, a cylindrical shaft disposed between the bottom end and the bulb shaped head; an absorbent element including at least one of a sponge, bead, gel, chemical compound, chemical composition, or medicament, the absorbent element further including a pulling cord member; a loading cylinder including a head, a shaft, and a base, the head of the loading cylinder being constructed and arranged to slidably engage with the receiving passage of the outer receptacle in an interlocking connection that al lows the head of the loading cylinder to slide within the receiving portion of the outer receptacle, wherein an upward sliding position the loading cylinder forces the absorbent element through the opening in the bulb shaped head with ease and comfort and wherein in a downward sliding position, to remove the loading cylinder from the outer receptacle.

Variation 19 may include the method as in variation 18 wherein the outer receptacle further includes a threaded base component defining a hand gripping portion.

Variation 20 may include the method as in any of variations 18 through 19 wherein the apparatus is configured to leave the outer receptacle and the absorbent element intact within a cavity while fluids within the cavity are absorbed by the absorbent element, the outer receptacle being configured to guide fluids towards the absorbent element and through the plurality of ergonomic arc-shaped members of the bulb-shaped head and its defined opening.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A device configured to be inserted into a vaginal cavity, the device comprising:
an outer receptacle having a bottom end, an expulsion end, and a cylindrical shaft portion disposed between the bottom end and the expulsion end, the cylindrical shaft portion defining a receiving passage therethrough extending between the bottom end and the expulsion end wherein the expulsion end comprises a bulb shaped head defining a plurality of slits defining a plurality of arc-shaped flexible flap members defining an opening and wherein the outer receptacle and the bulb shaped head member comprise an ergonomic shape adapted to be slidably inserted into the vaginal cavity with ease and comfort;
an absorbent element comprising at least one of a sponge, bead, gel, chemical compound, chemical composition, or medicament, the absorbent element adapted to conform to the receiving passage portion of the cylindrical shaft portion, the absorbent element being slidably inserted via the bottom end and the absorbent element further comprising a pulling cord member; and
a loading cylinder member having an ergonomic shape slidably engaged with the receiving passage portion for forcing the absorbent element through the receiving passage of the cylindrical shaft portion of the outer receptacle and at least partially out of the opening defined by the bulb shaped head member.

2. The device of claim 1, wherein the bulb shaped head comprises a longest cross-sectional dimension greater than the diameter of the cylindrical shaft portion.

3. The device of claim 1, wherein the bulb shaped head comprises a cross-sectional area larger than that of the cross-sectional area of the cylindrical shaft portion.

4. The device of claim 1, wherein the absorbent element is configured to reduce a rate of risk for toxic shock syndrome.

5. The device of claim 1, wherein the absorbent element comprises at least one of a spermicide or contraceptive.

6. The device of claim 1, wherein the absorbent element comprises at least one of a medical chemical or medicament for disease prevention.

7. The device of claim 1, wherein the device is configured to leave the outer receptacle and the absorbent element intact within the vaginal cavity.

8. The device of claim 1, wherein the outer receptacle is formed according to at least one of a selective predetermined length of the absorbent element and a selective predetermined radius of the absorbent element wherein the selective predetermined length is directly proportional with the absorbency rate of the absorbent element.

9. The device of claim 1, wherein the absorbent element is constructed and arranged to absorb at least one fluid at an effective absorption rate.

10. The device of claim 1, wherein the absorbent element absorbs the fluids at an absorption rate greater than fifty percent.

11. A method, comprising:
inserting an absorbent element comprising a cord into a receiving passage defined by an outer receptacle and sliding a loading cylinder within the receiving passage of the outer receptacle to position the absorbent element within the receiving passage to provide a feminine hygiene product adapted for insertion within a vaginal cavity;
inserting the feminine hygiene product into a cavity;
compressing the loading cylinder within the cylindrical shaft portion to expel the absorbent element through an opening defined by a plurality of arc-shaped flexible flap members and into the cavity with a reduced force impact for ease and comfort, wherein the absorbent element comprises at least one of a sponge, bead, gel, chemical compound, chemical composition, or medicament; and
removing the loading cylinder and the outer receptacle and leaving the absorbent element intact within the cavity.

12. The method of claim 11, further comprising:
removing the absorbent element from the cavity by pulling the cord.

13. The method of claim 11, wherein the absorbent element is constructed and arranged to absorb bodily fluids.

14. The method of claim 11, wherein the outer receptacle further comprises a bulb shaped head and a threaded base component defining a hand gripping portion.

15. The method of claim 11, wherein the absorbent element is specifically configured to absorb seminal fluids.

16. The method of claim 11, wherein the absorbent element further comprises at least one of a spermicide, a contraceptive, or a medical chemical for disease prevention.

17. The method of claim 11, wherein the outer receptacle, the bulb-shaped head member, and the loading cylinder comprise ergonomic configurations.

18. An apparatus adapted for use with a vaginal cavity, the apparatus comprising:
an outer receptacle having an ergonomic configuration, the outer receptacle defining a receiving passage and comprising a bottom end, the bottom end being opposite a bulb shaped head comprising a plurality of ergonomic arc-shaped members defining an opening therethrough in communication with the receiving passage, a cylindrical shaft disposed between the bottom end and the bulb shaped head;
an absorbent element comprising at least one of a sponge, bead, gel, chemical compound, chemical composition, or medicament, the absorbent element further comprising a pulling cord member; and
a loading cylinder comprising a head, a shaft, and a base, the head of the loading cylinder being constructed and arranged to slidably engage with the receiving passage of the outer receptacle in an interlocking connection that the head of the loading cylinder to slide within the receiving portion of the outer receptacle, wherein when in an upward sliding position the loading cylinder forces the absorbent element through the opening in the bulb shaped head with ease and comfort and wherein in a downward sliding position to remove the loading cylinder from the outer receptacle.

19. The apparatus of claim 18, wherein the outer receptacle further comprises a threaded base component defining a hand gripping portion.

20. The apparatus of claim 19, wherein the apparatus is configured to leave the outer receptacle and the absorbent element intact within a cavity while fluids within the cavity are absorbed by the absorbent element, the outer receptacle being configured to guide fluids towards the absorbent element and through the plurality of ergonomic arc-shaped members of the bulb-shaped head and the opening.
